(19) United States
(12) Patent Application Publication
(10) Pub. No.: US 2026/0128139 A1
(43) Pub. Date: May 7, 2026

(54) DATA GENERATION DEVICE, DATA GENERATION METHOD, AND RECORDING MEDIUM

(71) Applicant: NEC Corporation, Tokyo (JP)

(72) Inventors: Masanori Tsujikawa, Tokyo (JP); Remi Numajiri, Tokyo (JP); Satoshi Oda, Tokyo (JP); Masahiro Kubo, Tokyo (JP)

(73) Assignee: NEC Corporation, Tokyo (JP)

(21) Appl. No.: 19/357,300

(22) Filed: Oct. 14, 2025

(30) Foreign Application Priority Data
Nov. 5, 2024 (JP) 2024-193598

(51) Int. Cl. G16H 15/00; G16H 10/60; G16H 20/00; G16H 50/20

(52) U.S. Cl. CPC G16H 15/00; G16H 10/60; G16H 20/00; G16H 50/20

(57) ABSTRACT

The data generation device includes an acquisition unit, a virtual patient data generation unit, a time-series data generation unit, and an output unit. The acquisition unit acquires data regarding a treatment of a patient. The virtual patient data generation unit generates data regarding treatment in each stage of the medical condition of the virtual patient based on the data regarding treatment of the patient. The time-series data generation unit generates time-series data regarding the medical condition of the virtual patient from the generated data regarding treatment of the virtual patient. The output unit outputs the generated time-series data regarding the medical condition of the virtual patient. With such a configuration, the data generation device can support decision-making regarding the patient’s medical condition.

This application is based upon and claims the benefit of priority from Japanese Patent Application No. 2024-193598, filed on November 5, 2024 the disclosure of which is incorporated herein in its entirety by reference.

### TECHNICAL FIELD

The present disclosure relates to a data generation device and the like.

### BACKGROUND ART

Time-series data regarding a patient's medical condition may be used for planning a clinical trial plan in a pharmaceutical company. Such data used for making a plan regarding the patient may be generated using an information processing system.

The information processing system of JP 2022-141322 A identifies a treatment policy based on guideline data relevant to a patient's disease or case. Then, the information processing system of JP 2022-141322 A displays the identified treatment policy.

### SUMMARY

An object of the present disclosure is to provide a data generation device and the like capable of easily generating time-series data regarding the medical condition of a patient.

A data generation device according to an aspect of the present disclosure includes an acquisition unit that acquires data regarding treatment of a patient, a virtual patient data generation unit that generates data regarding treatment in each stage of a medical condition of a virtual patient based on the data regarding treatment of the patient, a time-series data generation unit that generates time-series data regarding a medical condition of the virtual patient from the generated data regarding treatment of the virtual patient, and an output unit that outputs the generated time-series data regarding the medical condition of the virtual patient.

A data generation method according to an aspect of the present disclosure includes acquiring data regarding treatment of a patient, generating data regarding treatment in each stage of a medical condition of a virtual patient based on the data regarding treatment of the patient, generating time-series data regarding a medical condition of the virtual patient from the generated data regarding treatment of the virtual patient, and outputting the generated time-series data regarding the medical condition of the virtual patient.

A non-transitory recording medium according to an aspect of the present disclosure stores a program for causing a computer to execute a process of acquiring data regarding treatment of a patient, a process of generating data regarding treatment in each stage of a medical condition of a virtual patient based on the data regarding treatment of the patient, a process of generating time-series data regarding a medical condition of the virtual patient from the generated data regarding treatment of the virtual patient, and a process of outputting the generated time-series data regarding the medical condition of the virtual patient.

### BRIEF DESCRIPTION OF THE DRAWINGS

Exemplary features and advantages of the present disclosure will become apparent from the following detailed description when taken with the accompanying drawings in which:

FIG. 1 is a diagram illustrating an example of a configuration of a data generation system in the present disclosure;

FIG. 2 is a diagram illustrating an example of a configuration of the data generation device in the present disclosure;

FIG. 3 is a diagram schematically illustrating an example of processing of generating data regarding treatment of a virtual patient in the present disclosure;

FIG. 4 is a diagram schematically illustrating an example of processing of generating data regarding treatment of a virtual patient in the present disclosure;

FIG. 5 is a diagram illustrating an example of a display screen of time-series data regarding a medical condition in the present disclosure;

FIG. 6 is a diagram illustrating an example of a display screen of time-series data regarding a medical condition in the present disclosure;

FIG. 7 is a diagram illustrating an example of a display screen of time-series data regarding a medical condition in the present disclosure;

FIG. 8 is a diagram illustrating an example of an operation flow of the data generation device in the present disclosure; and

FIG. 9 is a diagram illustrating an example of a hardware configuration of the data generation device in the present disclosure.

### EXAMPLE EMBODIMENT

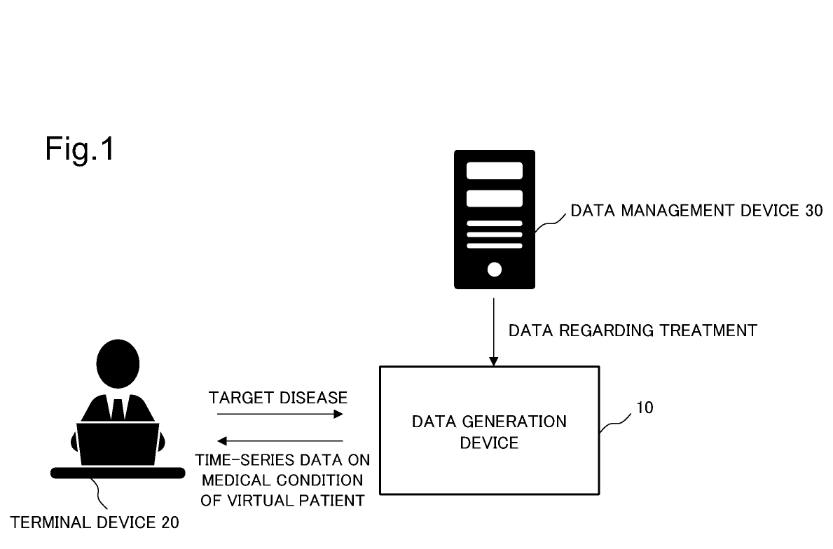

Example embodiments of the present disclosure will be described in detail with reference to the drawings. FIG. 1 is a diagram illustrating an example of a configuration of a data generation system. The data generation system includes a data generation device 10, a terminal device 20, and a data management device 30. The data generation device 10 is connected to the terminal device 20 via, for example, a network. The data generation device 10 is connected to the data management device 30 via, for example, a network. A plurality of terminal devices 20 and a plurality of data management devices 30 may be provided. The number of terminal devices 20 and the number of data management devices 30 may be appropriately set.

The data generation system generates, for example, time-series data regarding a medical condition of a patient. For example, the data generation system generates time-series data regarding the medical condition of a virtual patient. The virtual patient is, for example, a virtual person who is assumed to be suffering from a target disease to be subjected to generation of time-series data regarding the medical condition. The data regarding treatment of the virtual patient is, for example, data generated by the data generation system. The virtual patient is, for example, a person who is highly likely to be diagnosed as suffering from a target disease in a case where a specialist views data regarding treatment of the virtual patient generated by the data generation system.

The time-series data regarding the medical condition of the virtual patient is, for example, data regarding the patient’s medical condition at each stage on a time series from the discovery of the disease to the completion of the treatment. The time-series data regarding the medical condition of the virtual patient may be data regarding the patient’s medical condition at each stage on a time series from the hospitalization to the discharge from the hospital. The discovery of a disease may also include, for example, recognition of an abnormality of the body by the patient. Completion of treatment can also include, for example, the end of treatment at the end of life or the death of the patient.

For example, if a virtual patient is found with lung cancer at stage 0, progresses to stage 3, and then dies, the time-series data regarding the medical condition of the virtual patient is data regarding the medical condition of the patient at each stage between stages 0 to 3. The time-series data regarding the medical condition of the patient may be the number of patients at each stage of the medical condition.

The time-series data regarding the medical condition of the virtual patient is, for example, data indicating at least one of the condition and the treatment content of the patient at each stage of the medical condition. The patient’s condition is, for example, data of one or more items of a test result, findings of a medical worker, a chief complaint of the patient, and a feeling of the patient. The medical worker is, for example, a doctor, a nurse, a pharmacist, a laboratory technician, a clinical therapist, a psychotherapist, a caregiver, a consultant, or a clerical staff of a medical institution. The medical worker is not limited to the above. The patient’s condition is not limited to the above. The treatment content is, for example, data of one or more items of diagnosis, examination, medication, rehabilitation, counseling, physical care, hospital ward, equipment to be installed, and meal performed on a patient by a medical worker. The treatment content is not limited to the above. The time-series data regarding the patient’s medical condition may be a medical care cost at each stage of the medical condition. The time-series data regarding the patient’s medical condition is not limited to the above.

Stages of medical condition are, for example, stages of disease progression or treatment progression. For example, a stage of a medical condition is a stage of the progression of a medical condition or the progression of a treatment. For example, in a case where the stage of the medical condition is the progression of the medical condition, and the degree of progression of the medical condition is a disease represented with the stage, the stage of the medical condition is the stage. In a case where the stage of the medical condition is the stage of the progression of the treatment, the stage of the medical condition is, for example, the stages of visit, hospitalization, surgery, rehabilitation, and discharge. In a case where the stage of the medical condition is the stage of the progression of the treatment, the stage of the medical condition may be recognition of the abnormality of the body by the patient, information collection, visit, diagnosis, treatment, and support. The stage of the medical condition is not limited to the above.

The data generation system generates data regarding treatment of the virtual patient at each stage of the medical condition, for example, based on the data regarding treatment of the patient. Then, the data generation system generates time-series data regarding the patient’s medical condition based on the generated data regarding treatment of the virtual patient. The data regarding treatment of the virtual patient is, for example, virtual data generated by the data generation system. For example, the data regarding treatment of the virtual patient is data described in the electronic medical record as a record of treatment in a case where the virtual patient actually exists. Specific examples of the data regarding treatment of the virtual patient will be described later.

The data generation system generates data regarding treatment of the virtual patient using, for example, a generative model. The generative model is, for example, a machine learning model that generates data regarding treatment of the virtual patient using the data regarding treatment of the patient as an input. A specific example of the generative model will be described later. For example, the data generation system can generate the time-series data regarding the patient’s medical condition even in the case of a rare disease by generating the time-series data regarding the medical condition of the virtual patient based on the generated data regarding treatment of the virtual patient.

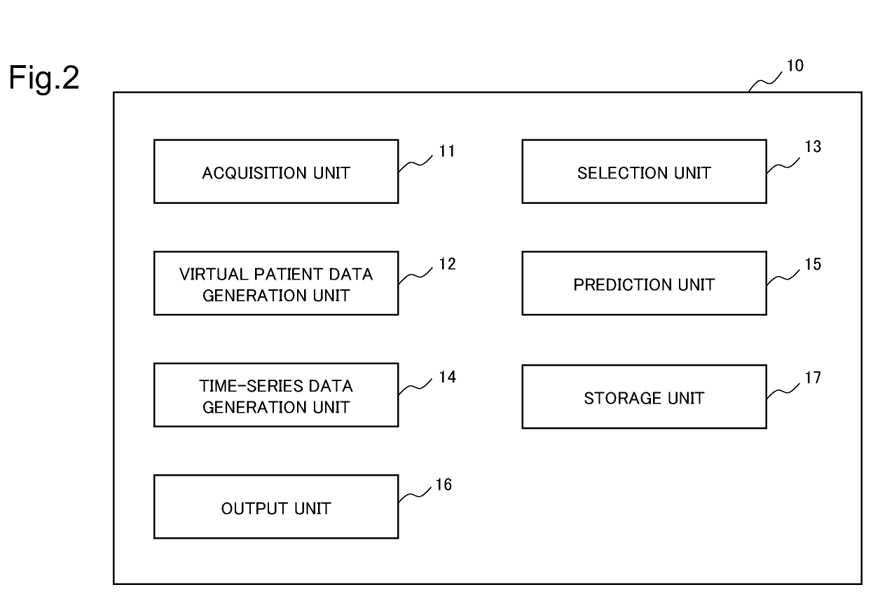

Here, an example of a configuration of the data generation device 10 will be described. FIG. 2 illustrates an example of a configuration of the data generation device 10. The data generation device 10 includes, as a basic configuration, an acquisition unit 11, a virtual patient data generation unit 12, a time-series data generation unit 14, and an output unit 16. The data generation device 10 further includes, for example, a selection unit 13, a prediction unit 15, and a storage unit 17.

The acquisition unit 11 acquires data regarding a treatment of a patient. The data regarding treatment of the patient is used, for example, to generate data regarding treatment of the virtual patient. The data regarding treatment of the patient is, for example, the treatment data of the patient in the medical institution. The treatment data of the patient is, for example, a record of a medical practice performed on the patient. Patient treatment data is, for example, a record of one or more items of diagnosis, examination, medication, surgery, follow-up, and patient’s condition. The patient’s condition includes, for example, information of one or more items of disease information, complication information, biomarkers, disease status, guideline scores, therapeutic effects, and test results. The therapeutic effect is, for example, an effect by the treatment, administration of a drug, and surgery. The effect is not limited to the above. The test result is, for example, a result of a biological test, a diagnostic imaging test, and a genome test. The test result is not limited to the above. The treatment data of the patient may include information about a person in charge who has performed the medical practice. The information about the person in charge who has performed the medical practice is, for example, information indicating a medical worker who has performed the medical practice on the patient. The information indicating the medical worker who has performed the medical practice on the patient is, for example, a name or an identifier of a doctor, a nurse, a pharmacist, and a physical therapist.

The acquisition unit 11 acquires, for example, data regarding the electronic medical record as data regarding treatment of a patient. The data regarding treatment of the patient may be examination data. In a case where the data regarding treatment of the patient is examination data, the data regarding treatment of the patient may be image data for image diagnosis. The data regarding treatment of the patient may be the data described in the receipt. The data regarding treatment may be, for example, recognition of a medical condition by a patient.

The acquisition unit 11 may acquire data regarding the health condition of the patient before visiting a hospital as data regarding treatment of the patient. For example, the acquisition unit 11 acquires data regarding treatment of a patient in a case where the patient visits another hospital as data regarding the health condition of the patient before visiting a hospital. The acquisition unit 11 may acquire, as the data regarding the health condition of the patient before visiting a hospital, information indicating the content recognized by the patient or a person around the patient with respect to the physical condition of the patient as the data regarding treatment of the patient. For example, in a case where the patient feels pain in a specific body part, the acquisition unit 11 acquires information indicating the body part where the pain has occurred, the period in which the pain has occurred, and the degree of pain as data regarding treatment of the patient.

The acquisition unit 11 may further acquire data regarding the health condition of the patient after discharge as data regarding treatment of the patient. The acquisition unit 11 acquires, for example, data regarding the health condition of a patient at home or a medical care facility after discharge as data regarding treatment of the patient. The medical care facility is, for example, a long-term care health facility, a long-term care medical hospital, or a special medical care facility for the elderly. The medical care facility is not limited to the above. The acquisition unit 11 may acquire data regarding treatment of a patient in a destination medical institution. The data regarding treatment of a patient is not limited to the above.

The acquisition unit 11 may acquire data regarding treatment of a subject patient whose future condition is to be predicted. For example, the acquisition unit 11 acquires data regarding treatment of the patient up to a time point at which the patient performs processing regarding prediction of the future condition. The acquisition unit 11 acquires data regarding treatment of a patient from the data management device 30, for example. The acquisition unit 11 may acquire data regarding treatment of a patient from the terminal device 20.

The acquisition unit 11 acquires, for example, information designating a target disease. The target disease is, for example, a disease for which time-series data regarding the medical condition of a virtual disease is to be generated. For example, in a case where the time-series data is used to grasp the medical condition of a trial patient, the target disease to be used with the time-series data is a disease to be used with a medicine for carrying out the trial. The acquisition unit 11 acquires, for example, information for designating the target disease, from the terminal device 20.

The virtual patient data generation unit 12 generates data regarding treatment in each stage of the medical condition of the virtual patient based on the data regarding treatment of the patient. For example, the virtual patient data generation unit 12 generates data regarding treatment at each stage of the medical condition for a virtual patient suffering from the target disease. The virtual patient data generation unit 12 generates, for example, data regarding treatment in each stage of the medical condition of the virtual patient using the generative model. The generative model is, for example, a machine learning model that generates data regarding treatment at each stage of a medical condition from data regarding treatment of a patient. The virtual patient data generation unit 12 generates data regarding treatment of each of the plurality of virtual patients using, for example, the generative model.

For example, the virtual patient data generation unit 12 generates data regarding treatment of the virtual patient using a language model as a generative model. For example, a large-scale language model is used as the language model. For example, as the language model, for example, Generative Pre-trained Transformer-2 (GPT-2), GPT-3, GPT-3.5, or GPT-4 can be used. Claude3, Claude3.5, text-to-text transfer transformer (T5), bidirectional encoder representations from transformers (BERT), robustly optimized BERT approach (RoBERTa), or efficiently learning an encoder that classifies token replacements accurately (ELECTRA) may be used as the language model. The language model used for generating the data regarding treatment of the virtual patient is not limited to the above.

For example, the virtual patient data generation unit 12 generates data regarding treatment of the virtual patient by using a prompt for instructing generation of data regarding treatment of the virtual patient as an input of the language model. The prompt to instruct the generation of data regarding treatment of the virtual patient includes, for example, an instruction to generate data regarding treatment of the virtual patient for each stage of the medical condition. The prompt for instructing the generation of the data regarding treatment of the virtual patient includes, for example, the name of the disease affected by the virtual patient to be generated. The disease name afflicted by the virtual patient to be generated is, for example, information designating the target disease. The prompt may include attributes of the virtual patient to be generated.

In a case where the electronic medical record is generated as the data regarding treatment of the virtual patient, the virtual patient data generation unit 12 generates the data of the electronic medical record of the virtual patient as the data regarding treatment of the virtual patient, for example, with a prompt including information indicating that the electronic medical record of the virtual patient is generated as an input of the language model. For example, the virtual patient data generation unit 12 generates data regarding treatment of a virtual patient with a prompt “Please create an electronic medical record of a virtual patient suffering from stage 3 liver cancer” as an input of a language model. The information indicative of generating the electronic medical record of the virtual patient may include one or more items of attributes of the virtual patient, the disease afflicted, the number of virtual patients, and the stage of the medical condition for which the electronic medical record is to be generated.

The virtual patient data generation unit 12 may generate data regarding treatment for each stage of the medical condition for one virtual patient. For example, the virtual patient data generation unit 12 generates data regarding treatment of a virtual patient suffering from stage 3 liver cancer for each of stage 0, stage 1, stage 2, and stage 3. For example, the virtual patient data generation unit 12 generates data of the electronic medical record in stage 0 of the medical condition with a prompt “Please create electronic medical record data for stage 0 virtual patient suffering from stage 3 liver cancer” as an input of the language model. The virtual patient data generation unit 12 generates data of the electronic medical record in each of the stages where the medical condition is stage 1, stage 2, and stage 3, for example. Then, the virtual patient data generation unit 12 generates the electronic medical record including the description in each stage of the medical condition from stage 0 to stage 3 using, for example, the data of the electronic medical record in which the medical condition is from stage 0 to stage 3.

The virtual patient data generation unit 12 generates, for example, data regarding treatment for a predetermined number of people. For example, the predetermined number of people is set to be a sufficient number for grasping the progression of the medical condition for each target disease. The predetermined number of people may be set to be a sufficient number for grasping the progression of the medical condition for each attribute of the virtual patient. The predetermined number of people may be set to be a sufficient number for grasping the progression of the medical condition for each stage of the medical condition to be finally reached. The predetermined number of people may be set to be a sufficient number for grasping the progression of the medical condition for each stage of the medical condition in a case where it is confirmed that the patient suffers from the disease.

In a case where the data regarding treatment of the virtual patient is generated using the language model, the virtual patient data generation unit 12 may generate the data regarding treatment of the virtual patient by a RAG (Retrieval Augmented Generation) technology using the data regarding treatment of the patient as a data source. For example, the virtual patient data generation unit 12 uses the electronic medical record of the patient as the data source as the data regarding treatment of the patient, and generates the data of the electronic medical record of the virtual patient as the data regarding treatment of the virtual patient.

In a case of generating the electronic medical record of the virtual patient suffering from the rare disease, the virtual patient data generation unit 12 generates the electronic medical record of the virtual patient by using, for example, data regarding treatment of the patient actually suffering from the rare disease as a data source. The rare disease is, for example, a disease in which there are few patients who develop the disease. That is, the rare disease is, for example, a disease in which there is not sufficient data regarding treatment of the patient. In a case of generating the electronic medical record of the virtual patient suffering from the rare disease, the virtual patient data generation unit 12 may generate the electronic medical record of the virtual patient, for example, by further using data regarding treatment of a patient actually suffering from a disease similar to the rare disease as a data source. A similar disease refers to, for example, at least the same type of disease, occurrence factor, and occurrence site.

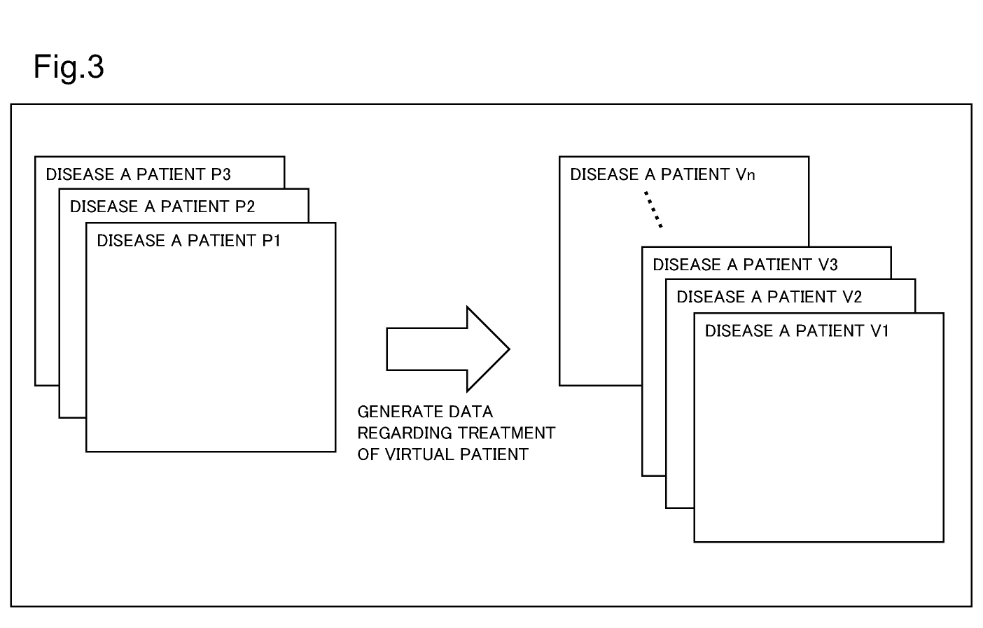

FIG. 3 is a diagram schematically illustrating an example of processing of generating data regarding treatment of a virtual patient. In the example of FIG. 3, electronic medical records of n virtual patients suffering from a disease A are generated using the electronic medical records of the patients P1, P2, and P3 suffering from the disease A as a data source. For example, the virtual patient data generation unit 12 generates the electronic medical record of the virtual patient as an input to a language model that uses a prompt including an instruction to generate the electronic medical record of the virtual patient as a generative model.

The virtual patient data generation unit 12 may generate data regarding complications of the virtual patient as data regarding treatment of the virtual patient. The virtual patient data generation unit 12 generates data of the electronic medical record of the virtual patient who has developed the complication, for example, using a prompt including the name of each disease included in the complication as the information indicating that the electronic medical record of the virtual patient is generated.

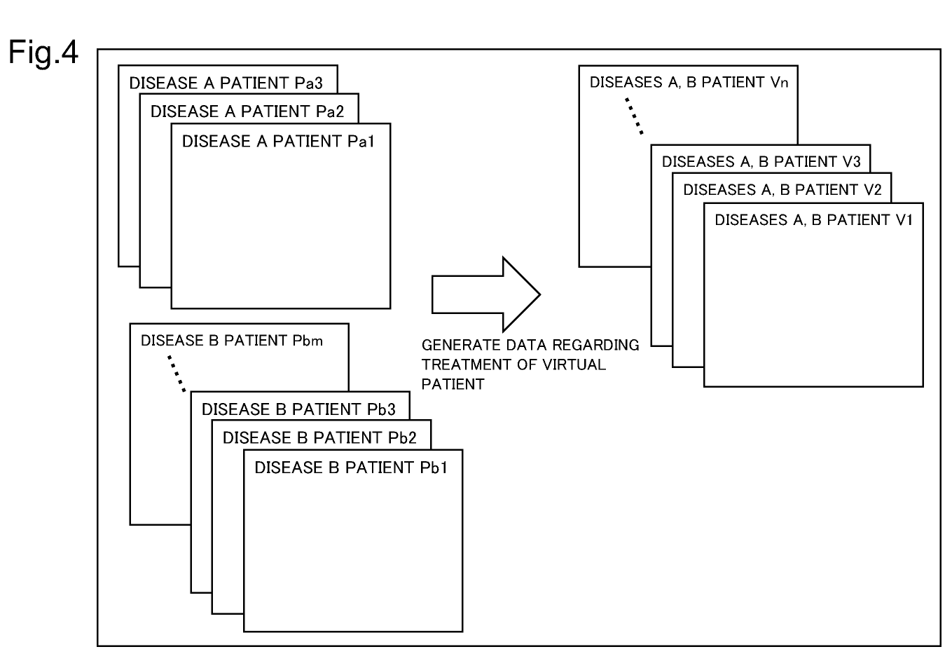

FIG. 4 is a diagram schematically illustrating an example of processing of generating data regarding treatment of a virtual patient suffering from complications. The example of FIG. 4 shows a process for generating data regarding treatment of a virtual patient suffering from a disease A as well as a disease B. In the example of FIG. 4, electronic medical records of patients Pa1, Pa2, and Pa3 suffering from the disease A and electronic medical records of m patients suffering from the disease B are used as data sources to generate electronic medical records of n virtual patients suffering from the diseases A and B. For example, the virtual patient data generation unit 12 generates the electronic medical record of the virtual patient as an input of a language model using a prompt including an instruction to generate the electronic medical record of the virtual patient suffering from the diseases A and B as a generative model.

The virtual patient data generation unit 12 may generate, for example, data regarding treatment in each stage of the medical condition of the virtual patient, the medical condition including a patient’s condition before visiting a hospital. The virtual patient data generation unit 12 generates data regarding treatment of the virtual patient, for example, using data indicating the health condition of the patient before visiting a hospital as a data source. The data indicating the health condition of the patient before visiting a hospital is, for example, data of a medical examination. The data indicating the health condition of the patient before visiting a hospital may include at least one of recognition of the patient with respect to his/her physical condition and recognition of the patient's physical condition by surrounding people. The data indicating the health condition of the patient before visiting a hospital may be, for example, data regarding treatment in another medical institution that the patient has visited before visiting a hospital.

The virtual patient data generation unit 12 may generate data regarding treatment in each stage of the medical condition of the virtual patient, the medical condition including a patient’s condition after discharge. The virtual patient data generation unit 12 generates data regarding treatment of the virtual patient, for example, using data indicating the health condition of the patient after discharge as a data source. The data indicating the health condition of the patient after discharge is, for example, data of a medical examination. The data indicating the health condition of the patient after discharge may be, for example, an examination result in an outpatient clinic. The data indicating the health condition of the patient after discharge may include at least one of recognition of the patient with respect to the physical condition of the patient and recognition by surrounding persons of the patient with respect to the physical condition of the patient. The data indicating the health condition of the patient after discharge may be, for example, data regarding treatment in another medical institution.

The virtual patient data generation unit 12 may generate data regarding treatment of the virtual patient using the statistical data of the data regarding treatment. For example, the virtual patient data generation unit 12 generates data regarding treatment of the virtual patient by replacing the data of a predetermined item among the data regarding treatment such that the value of the data of the predetermined item is included in the statistical data. For example, the virtual patient data generation unit 12 generates data regarding treatment of the virtual patient by replacing data of a predetermined item based on, for example, an average value, a mode value, or a median value of data regarding treatment of the patient and a variance. As the statistical data of the data regarding treatment, for example, data regarding treatment of a patient suffering from a disease similar to the target disease is used. As the statistical data of the data regarding treatment, data regarding treatment of a patient suffering from a target disease may be used.

The predetermined item is set, for example, for each target disease. The predetermined item is, for example, an item that is greatly affected by the disease among the data included in the data regarding treatment. The item is, for example, a type of data. For example, in a case where the data is the measurement results of the blood glucose level and the uric acid level, each of the blood glucose level and the uric acid level is relevant to an item. The predetermined item may be an item that requires attention in treating a disease among items included in the data regarding treatment. Among the predetermined items included in the data regarding treatment, the item may have a low relationship with the disease although attention is required for the treatment of the influence.

The virtual patient data generation unit 12 may generate data regarding treatment of the virtual patient using an image generative model that generates image data for image diagnosis as a generative model. For example, the virtual patient data generation unit 12 generates image data for image diagnosis for each stage of the medical condition using the image generative model. Then, the virtual patient data generation unit 12 generates data regarding treatment of the virtual patient by determining the medical condition based on the image data for image diagnosis for each stage of the medical condition, for example. For example, in a case where the image data for image diagnosis is data of a computed tomography (CT) examination for examining a tumor, the image generative model generates a plurality of pieces of image data in which the size of the tumor shown in the image of the same tumor is different. For example, the image generative model generates a plurality of pieces of image data in which the tumor grows. The image generative model may generate a plurality of pieces of image data in which the tumor shrinks as a result of the treatment. The image generative model may generate an image in which a new tumor has occurred at a location different from the existing tumor. Then, the virtual patient data generation unit 12 generates data regarding treatment in each stage of the medical condition based on the size of the tumor shown in the image generated by the image generative model, for example.

The image generative model is generated by machine learning using, for example, a generative adversarial network (GAN). Algorithm for generating the image generative model is not limited to the above. The image generative model is generated by, for example, a device outside the data generation device 10. The generative model may be a machine learning model capable of processing both language data and image data.

The data regarding treatment of the virtual patient may be generated in an information processing apparatus outside the data generation device 10. In this case, the virtual patient data generation unit 12 outputs a request for generating data regarding treatment of the virtual patient to an external information processing apparatus in which the generative model operates, for example. Then, the virtual patient data generation unit 12 acquires data regarding treatment of the virtual patient from, for example, an external information processing apparatus in which the generative model operates.

In a case where the processing of generating the data regarding treatment of the virtual patient is performed in a device outside the data generation device 10, the virtual patient data generation unit 12 outputs a prompt including information indicating that the electronic medical record of the virtual patient is to be generated to an information processing apparatus in which a language model that performs processing of generating the data regarding treatment of the virtual patient based on the prompt operates, for example. Then, the virtual patient data generation unit 12 acquires the generated data regarding treatment of the virtual patient from the information processing apparatus that has generated the data regarding treatment of the virtual patient, for example.

For example, the selection unit 13 selects data suitable as the target disease data among the data regarding treatment of the virtual patient generated by the virtual patient data generation unit 12. For example, the selection unit 13 selects data regarding treatment of the virtual patient to be used for extracting the time-series data regarding the medical condition of the virtual patient based on the suitability degree of the data regarding treatment of the virtual patient generated by the virtual patient data generation unit 12. The suitability degree is, for example, an index indicating suitability as data to be used for extracting the time-series data regarding the medical condition of the virtual patient.

For example, the selection unit 13 calculates the suitability degree of the data regarding treatment of the virtual patient generated by the virtual patient data generation unit 12. Then, the selection unit 13 selects the data regarding treatment of the virtual patient whose calculated suitability degree satisfies the predetermined reference as the data regarding treatment of the virtual patient used for extracting the time-series data regarding the medical condition of the virtual patient. The predetermined reference is set such that, for example, in a case where the suitability degree satisfies the reference, the data regarding treatment of the virtual patient satisfying the reference is set to be appropriate data as the data of the patient with the target disease.

As the suitability degree, for example, a similarity between the reference data and the generated data regarding treatment of the virtual patient is used. The similarity is, for example, an index indicating the degree of coincidence between the reference data of each disease and the data regarding treatment of the virtual patient. The reference data of each disease is set based on, for example, an assumed value of data regarding treatment in a case where a patient suffers from the disease. In a case where the data regarding treatment of the virtual patient is the electronic medical record, the similarity may be the similarity between the electronic medical record of the patient actually suffering from the target disease and the electronic medical record of the virtual patient.

For example, the selection unit 13 calculates the similarity of the data regarding treatment of the virtual patient with respect to the target disease using a similarity calculation model. The similarity calculation model is, for example, a machine learning model that calculates the similarity of data regarding treatment of the virtual patient with respect to the target disease from data regarding treatment of the virtual patient. The similarity calculation model converts, for example, data of an item included in the reference data among the reference data of the target disease and the data regarding treatment of the virtual patient into a feature vector. Then, the selection unit 13 calculates, for example, similarity between the converted feature vectors. For example, a Euclidean distance or a cosine similarity is used as the similarity between the feature vectors. The similarity calculation model is generated, for example, in a device outside the data generation device 10.

The suitability degree may be an index based on a score set for each piece of data regarding treatment. For example, the selection unit 13 calculates the score of each piece of data regarding treatment of the virtual patient based on the score reference set for each piece of data regarding treatment. For example, the selection unit 13 calculates the total value of the scores as the suitability degree of the data regarding treatment of each virtual patient. Then, the selection unit 13 selects the data regarding treatment of the virtual patient whose calculated suitability degree satisfies the predetermined reference as the data regarding treatment of the virtual patient used for extracting the time-series data regarding the medical condition of the virtual patient. The method for calculating the suitability degree is not limited to the above.

The selection unit 13 may select data regarding treatment of the virtual patient for the target disease using a selection model. The selection model is, for example, a machine learning model that determines whether the data regarding treatment of the virtual patient is appropriate as the data regarding treatment of the patient with the target disease. For example, the selection unit 13 selects, as the data regarding treatment of the virtual patient, data regarding treatment of the virtual patient determined to be suitable as the data regarding treatment of the patient with the target disease by the selection model. The selection model is generated by learning a relationship between the selection reference and the data regarding treatment of the virtual patient and suitability as data regarding treatment of the patient with the target disease. The selection model is generated by deep learning using a neural network, for example. A machine learning algorithm for generating the selection model is not limited to the above. The selection model is generated, for example, by a device outside the data generation device 10.

The time-series data generation unit 14 generates time-series data regarding the medical condition of the virtual patient from the generated data regarding treatment of the virtual patient. The time-series data regarding the medical condition of the virtual patient is, for example, data in a time-series order for at least one of the treatment content to be performed on the patient and the patient’s condition. The time-series data generation unit 14 generates, for example, data in the form of a patient journey as time-series data regarding the medical condition of the virtual patient. For example, the time-series data generation unit 14 extracts data in each stage of the medical condition from the data regarding treatment of the virtual patient, and generates data in a time-series order as time-series data regarding the medical condition of the virtual patient.

For example, in a case where the stage of the medical condition is set by the stage, the time-series data generation unit 14 extracts data regarding the medical condition in each stage from the data regarding treatment of the virtual patient, and generates data of the stage order. For example, in a case where the stage of the medical condition is classified into visit, hospitalization, surgery, rehabilitation, and discharge, and each stage is in the above order on a time series, the time-series data generation unit 14 extracts data regarding the medical condition in each stage in the above order from the data regarding treatment of the virtual patient, and generates data in a time-series order as time-series data regarding the medical condition of the virtual patient.

For example, the time-series data generation unit 14 generates treatment contents in each stage of a medical condition as time-series data regarding the medical condition. The time-series data generation unit 14 generates, for example, information indicating treatment to be performed on a patient at each stage of a medical condition by each medical worker as time-series data regarding the medical condition. For example, in a case where the medical worker is a doctor, the time-series data generation unit 14 generates, for example, information indicating a treatment action, medication, and examination performed by the doctor on a patient at each stage of a medical condition. For example, in a case where the medical worker is a nurse, the time-series data generation unit 14 generates, for example, information indicating examinations, confirmation items, and advice to be performed by the nurse on the patient at each stage of the medical condition. The time-series data generation unit 14 may generate findings of the medical worker at each stage of the medical condition as time-series data regarding the medical condition. For example, the time-series data generation unit 14 generates findings of a doctor at each stage of a medical condition as time-series data regarding the medical condition.

For example, the time-series data generation unit 14 generates the state of the body at each stage of the medical condition as time-series data regarding the medical condition. The time-series data generation unit 14 generates, for example, information indicating findings of a doctor at each stage of a medical condition, treatment being performed, contents of medication, and a test result as time-series data regarding the medical condition. The time-series data generation unit 14 may generate a chief complaint of a patient at each stage of a medical condition as time-series data regarding the medical condition. The patient's chief complaint may include the patient's feeling at each stage of the medical condition.

The time-series data generation unit 14 may generate the number of patients in each stage of the medical condition as time-series data regarding the medical condition. For example, the time-series data generation unit 14 extracts the stage of the medical condition for each lapse of time from the time-series data regarding treatment of each of the plurality of virtual patients. Then, the time-series data generation unit 14 generates, for example, information indicating the ratio of the number of people in each stage of the medical condition as time-series data regarding the medical condition for each lapse of time. The ratio of the number of people in each stage of the medical condition is, for example, the number of virtual patients relevant to each stage of the medical condition with respect to the total number of virtual patients suffering from the target disease at a certain time point on a time series. The time-series data generation unit 14 may generate the number of people at each stage of the medical condition at each time point on a time series as the time-series data regarding the medical condition based on the number of people suffering from the target disease at the time point serving as the starting point.

For example, in a case where the target disease is lung cancer, the time-series data generation unit 14 generates the number of patients for each stage as time-series data regarding the medical condition. For example, the time-series data generation unit 14 extracts a stage for each lapse of time from time-series data regarding treatment of each of a plurality of virtual patients suffering from lung cancer. Then, the time-series data generation unit 14 generates, for example, information indicating the number of virtual patients relevant to each stage as time-series data regarding the medical condition for each lapse of time. The time-series data generation unit 14 may generate information indicating the ratio of the number of virtual patients relevant to each stage as time-series data regarding the medical condition for each lapse of time.

The prediction unit 15 predicts, for example, a transition of data regarding treatment of the subject patient. The subject patient is, for example, a patient to be treated with reference to time-series data regarding the medical condition of the virtual patient. The subject patient may be a subject of a clinical trial in a case where performing the clinical trial with reference to time-series data regarding the medical condition of the virtual patient. The prediction unit 15 extracts, for example, a virtual patient having similar data regarding treatment in the initial stage to the subject patient. The initial stage is, for example, a predetermined period from the start of treatment. The predetermined period is, for example, a period from the start of treatment until a full treatment policy is determined. The determination of a full treatment policy is, for example, determination of the presence or absence of surgery and determination of a treatment policy after surgery. The predetermined period is not limited to the above. For example, the prediction unit 15 extracts a virtual patient whose data regarding treatment in the initial stage is similar to the data regarding treatment of the subject patient in the initial stage based on the similarity between the data regarding treatment of the subject patient in the initial stage and the generated data regarding treatment of the virtual patient.

The prediction unit 15 predicts, for example, transition of data regarding treatment of the subject patient based on the extracted time-series data regarding the medical condition of the virtual patient. For example, the prediction unit 15 predicts the transition of the data regarding treatment of the subject patient assuming that the data regarding treatment of the subject patient transitions similarly to the extracted time-series data regarding the medical condition of the virtual patient. In a case where the subject patient is similar to a plurality of virtual patients in the data regarding treatment in the initial stage, the prediction unit 15 may predict the transition of the data regarding treatment of the subject patient for a plurality of patterns based on the time-series data regarding the medical condition of each similar virtual patient.

For example, the prediction unit 15 calculates similarity between the data regarding treatment of the subject patient in the initial stage and the generated data regarding treatment of the virtual patient using a calculation model. The calculation model is a machine learning model that calculates the similarity between the data regarding treatment of the subject patient in the initial stage and the generated data regarding treatment of the virtual patient. The calculation model converts, for example, data regarding treatment of the subject patient in the initial stage and the generated data regarding treatment of the virtual patient into feature vectors. Then, the prediction unit 15 calculates, for example, similarity between the converted feature vectors. For example, a Euclidean distance or a cosine similarity is used as the similarity between the feature vectors. The calculation model is generated, for example, by a device outside the data generation device 10.

The prediction unit 15 may predict the data regarding treatment of the subject patient in a stage after the initial stage using a prediction model based on the data regarding treatment of the subject patient in the initial stage. The prediction model is, for example, a machine learning model that predicts data regarding treatment in a stage after the initial stage on a time series from data of the initial stage of the patient. The prediction model is generated by deep learning using a neural network, for example. A machine learning algorithm for generating the prediction model is not limited to the above. The prediction model is generated, for example, by a device outside the data generation device 10. The prediction unit 15 may extract data regarding treatment of a virtual patient similar to the prediction result by the prediction model. For example, the prediction unit 15 extracts data regarding treatment of a virtual patient similar to the prediction result by the prediction model by a method similar to the case of extracting a virtual patient with similar data regarding treatment of the subject patient in the initial stage.

The output unit 16 outputs the generated time-series data regarding the medical condition of the virtual patient. The output unit 16 outputs, for example, at least one of the patient’s condition and the treatment content for each stage of the medical condition as time-series data regarding the medical condition of the virtual patient. The output unit 16 may output a graph of time-series data regarding the medical condition of each virtual patient. For example, the output unit 16 outputs time-series data regarding the medical condition of each virtual patient using a graph with the stage of the medical condition on the vertical axis and the lapse of time on the horizontal axis. The output unit 16 may output the number of virtual patients for each stage of the medical condition as time-series data regarding the medical condition of the virtual patient.

The output unit 16 may output a prediction result of data regarding treatment of the subject patient in a stage after the initial stage. The output unit 16 may output data regarding treatment of the virtual patient similar to the prediction result together with the prediction result. The output unit 16 outputs, for example, data regarding treatment of a virtual patient with similar data regarding treatment of the subject patient in the initial stage. The output unit 16 may output data regarding treatment of a plurality of virtual patients having similar data regarding treatment of the subject patient in the initial stage.

The output unit 16 outputs, for example, the generated time-series data regarding the medical condition of the virtual patient to the terminal device 20. The output unit 16 outputs the generated time-series data regarding the medical condition of the virtual patient to a display device (not illustrated) connected to the data generation device 10.

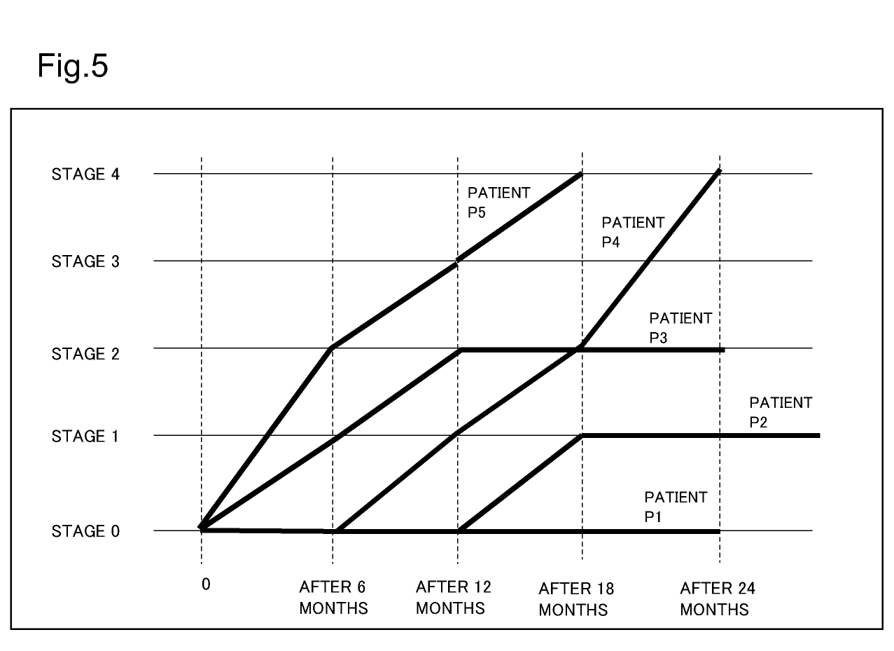

FIG. 5 is an example of a display screen displaying time-series data regarding a medical condition of a patient. The example of the display screen of FIG. 5 illustrates a change in the medical condition of each patient. In the example of the display screen of FIG. 5, for each of the virtual patients, the change in the stage of the medical condition for each lapse of time from the diagnosis of stage 0 cancer is illustrated. In the example of the display screen in FIG. 5, the horizontal axis of the graph indicates the lapse of time from the diagnosis of stage 0 cancer. In the example of the display screen of FIG. 5, the vertical axis of the graph indicates the stage of the medical condition of each virtual patient. By referring to the example of the display screen of FIG. 5, for example, it is possible to grasp the tendency of the change in the medical condition of each patient.

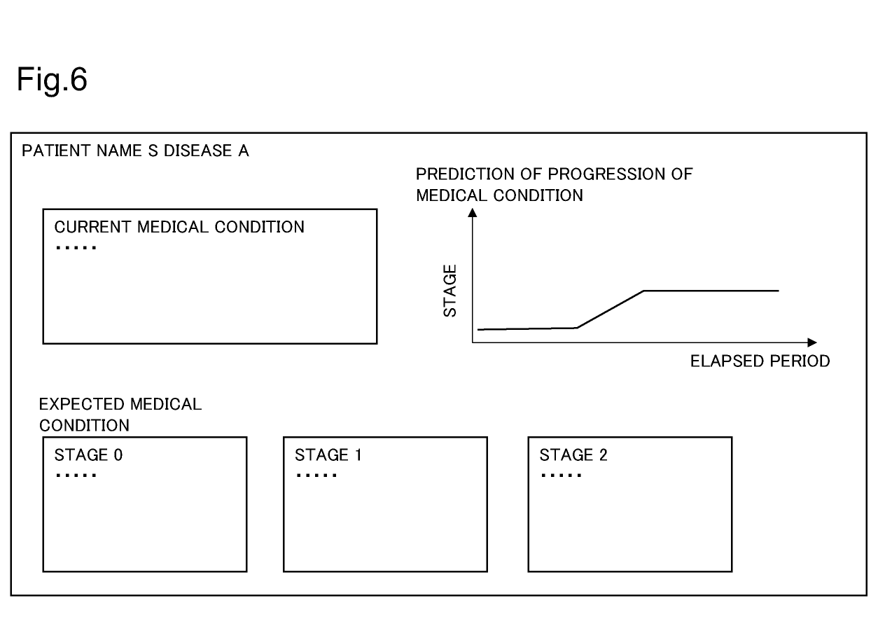

FIG. 6 is an example of a display screen for displaying a prediction result of a medical condition of a patient. In the example of the display screen of FIG. 6, the name of the patient and the name of the suffering disease are displayed at the top. In the example of the display screen of FIG. 6, graphs indicating the current patient’s medical condition and the prediction result of the progression of the medical condition are displayed. In the graph showing the prediction result of the progression of the medical condition, the horizontal axis indicates the lapse of time from the present, and the vertical axis indicates the stage of the medical condition. In the example of the display screen of FIG. 6, the expected medical condition is displayed for each stage. With reference to the example of the display screen of FIG. 6, the user of the data generation device 10 can create a treatment plan or a clinical trial plan, for example, based on the patient’s condition in a case where the medical condition progresses.

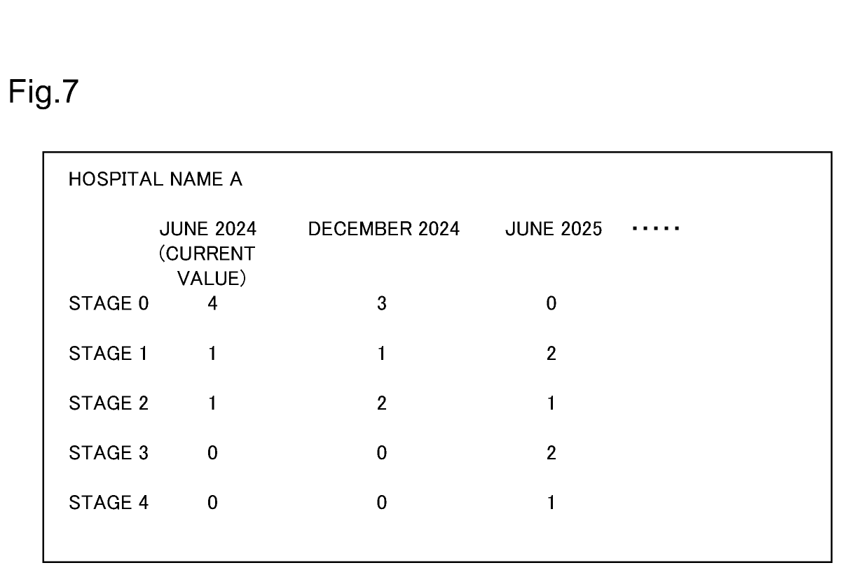

FIG. 7 is an example of a display screen displaying a prediction result of the number of patients for each stage of the medical condition. In the example of the display screen of FIG. 7, the name of the hospital for which the prediction is made is illustrated in the upper part. In the example of the display screen of FIG. 7, the prediction result of the number of patients for each stage of the medical condition in a case where the time has elapsed is illustrated. In the example of the display screen of FIG. 7, a prediction result of how the current number of patients for each stage of the medical condition indicated as the current value changes over time is illustrated. With reference to the example of the display screen of FIG. 7, the user of the data generation device 10 can create, for example, a patient acceptance plan or a clinical trial plan.

The storage unit 17 stores, for example, data regarding processing of generating time-series data regarding the medical condition of a virtual patient. The storage unit 17 stores, for example, data regarding treatment of a patient. The storage unit 17 stores, for example, data regarding treatment of a virtual patient. The storage unit 17 stores, for example, time-series data regarding the medical condition of the virtual patient. The storage unit 17 stores, for example, the generative model. The storage unit 17 stores, for example, a similarity calculation model. The storage unit 17 stores, for example, the selection model. The storage unit 17 stores, for example, the calculation model. The storage unit 17 stores, for example, the prediction model. The generative model, the similarity calculation model, the selection model, the calculation model, and the prediction model may be stored in a storage outside the data generation device 10.

The terminal device 20 is, for example, a terminal device used by a user in processing regarding extraction of time-series data regarding a medical condition of a virtual patient. The terminal device 20 outputs, for example, the time-series data regarding the medical condition of the virtual patient from the output unit 16 of the data generation device 10. Then, the terminal device 20 outputs the time-series data regarding the medical condition of the virtual patient to a display device (not illustrated), for example.

The terminal device 20 may acquire, from the output unit 16 of the data generation device 10, a prediction result of data regarding treatment of the subject patient in a stage after the initial stage. In a case where the prediction result of the data regarding treatment of the subject patient in a stage after the initial stage is acquired, the prediction result of the data regarding treatment of the subject patient in a stage after the initial stage is output to the terminal device 20, for example, a display device (not illustrated).

The terminal device 20 acquires, for example, information designating a target disease which is input by a user's operation. Then, for example, the terminal device 20 outputs information designating the target disease to the acquisition unit 11 of the data generation device 10.

The user is, for example, a person engaged in work related to healthcare. The user is, for example, a medical worker. The medical worker is, for example, a doctor, a nurse, a pharmacist, a laboratory technician, a clinical therapist, a psychotherapist, a caregiver, a consultant, or a clerical staff of a medical institution. The medical worker is not limited to the above.

In a case where the time-series data regarding the medical condition of the virtual patient is used for the clinical trial, the user is, for example, a person in charge of a medical institution or a person in charge of an institution that has been commissioned by the medical institution to conduct the clinical trial. The organization to which the hospital entrusts the handling of the clinical trial is, for example, a site management organization (SMO). The person in charge of an institution that has been commissioned by a medical institution to conduct the clinical trial is, for example, a clinical research coordinator (CRC). The terminal device 20 may be a terminal device used by a person in charge who performs a clinical trial in a pharmaceutical company or a person in charge in an institution entrusted with a clinical trial by a pharmaceutical company that performs a medicine clinical trial. The institution entrusted with the clinical trial by the pharmaceutical company is, for example, a contract research organization (CRO). The person in charge of the institution entrusted with the clinical trial by the pharmaceutical company is, for example, a clinical research associate (CRA). The terminal device is a device used by a person in charge of a medical institution or a person in charge of an institution commissioned by the medical institution to conduct the clinical trial. The organization to which the hospital entrusts the handling of the clinical trial is, for example, a site management organization (SMO). The person in charge of an institution that has been commissioned by a medical institution to conduct the clinical trial is, for example, a clinical research coordinator (CRC). The terminal device 20 may be a terminal device used by a person in charge who performs a clinical trial in a pharmaceutical company or a person in charge in an institution entrusted with a clinical trial by a pharmaceutical company that performs a medicine clinical trial. The institution entrusted with the clinical trial by the pharmaceutical company is, for example, a contract research organization (CRO). The person in charge of the institution entrusted with the clinical trial by the pharmaceutical company is, for example, a clinical research associate (CRA).

As the terminal device 20, for example, a personal computer, a tablet computer, a smartphone, or a smartwatch can be used. An information processing apparatus used for the terminal device 20 is not limited to the above.

The data management device 30 is a device that stores data regarding treatment of a patient. The data regarding treatment of the patient is, for example, data of an electronic medical record input by a doctor. The information in the electronic medical record may be data input by a nurse, a laboratory technician, a physical therapist, or a counselor. The data regarding treatment of the patient may be information of one or more items of patient disease information, complication information, biomarkers, disease status, guideline scores, medical histories, efficacy, and test results other than data described in the electronic medical record. The data management device 30 outputs data regarding treatment to the acquisition unit 11 of the data generation device 10, for example.

For example, the data management device 30 may save the data regarding treatment of a patient as anonymized information or pseudonymized information. The anonymized information is, for example, information processed in such a way that an individual cannot be identified even if the anonymized information is collated with other information. The pseudonymized information is, for example, information processed in such a way that an individual cannot be identified by itself but can be identified in a case where the information is collated with other information.

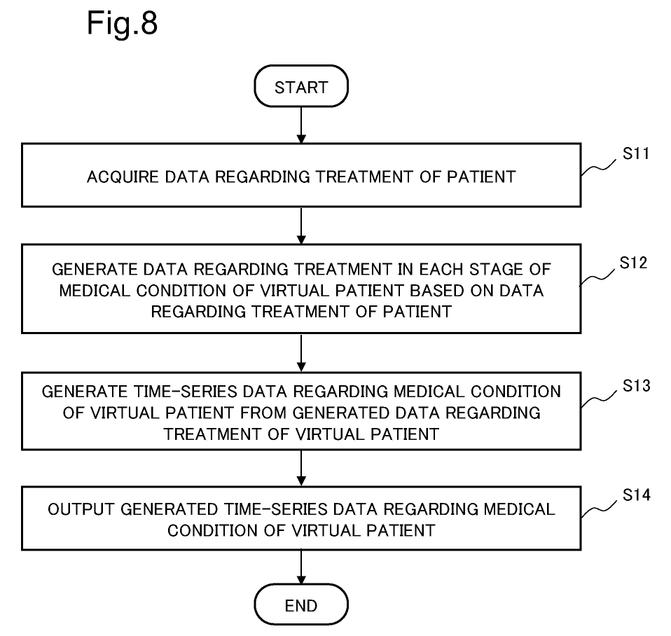

An operation of the data generation device 10 in a process of generating time-series data of data regarding a medical condition of a virtual patient will be described. FIG. 8 illustrates an example of a flow in a process in which the data generation device 10 generates time-series data of data regarding a medical condition of a virtual patient.

The acquisition unit 11 acquires data regarding treatment of a patient (step S11). The acquisition unit 11 acquires data regarding treatment of a patient from the data management device 30, for example.

In a case where the data regarding treatment of the patient is acquired, the virtual patient data generation unit 12 generates data regarding treatment in each stage of the medical condition of the virtual patient based on the data regarding treatment of the patient (step S12). The virtual patient data generation unit 12 generates, for example, data regarding treatment of each virtual patient.

In a case where the data regarding treatment of the virtual patient is generated, the time-series data generation unit 14 generates time-series data regarding the medical condition of the virtual patient from the generated data regarding treatment of the virtual patient (step S13).

In a case where the time-series data regarding the medical condition of the virtual patient is generated, the output unit 16 outputs the generated time-series data regarding the medical condition of the virtual patient (step S14). The output unit 16 outputs, for example, the time-series data regarding the medical condition of the virtual patient to the terminal device 20.

Each processing in the data generation device 10 may be executed in a distributed manner in a plurality of information processing apparatuses connected via a network. For example, the processing in the virtual patient data generation unit 12 and the selection unit 13 and the processing in the time-series data generation unit 14 may be performed in another information processing apparatus. Which information processing apparatus performs each processing in the data generation device 10 can be appropriately set.

The data generation device 10 generates data regarding treatment in each stage of the medical condition of the virtual patient based on the data regarding treatment of the patient. Then, the data generation device 10 generates time-series data regarding the medical condition of the virtual patient from the generated data regarding treatment of the virtual patient. As described above, the data generation device 10 generates the data regarding treatment of the virtual patient and generates the time-series data regarding the medical condition of the virtual patient from the generated data, thereby easily generating the time-series data regarding the patient’s medical condition.

Even in a case of a rare disease that is a disease with few cases, the data generation device 10 can easily generate the time-series data regarding the medical condition of the virtual patient assuming that the patient suffers from the rare disease, for example, by generating the time-series data regarding the medical condition based on the data regarding treatment of the virtual patient. Therefore, by referring to the time-series data regarding the medical condition of the virtual disease, for example, the medical worker can easily make a decision on treatment of a patient suffering from a rare disease. By referring to the time-series data regarding the medical condition of the virtual disease, for example, a person in charge of drug trials for rare diseases can improve the accuracy of clinical trial planning.

By generating the time-series data regarding the medical condition of the virtual patient whose data regarding treatment in the initial stage is similar to that of the subject patient, the data generation device 10 can facilitate, for example, grasping the transition of the medical condition of the subject patient.

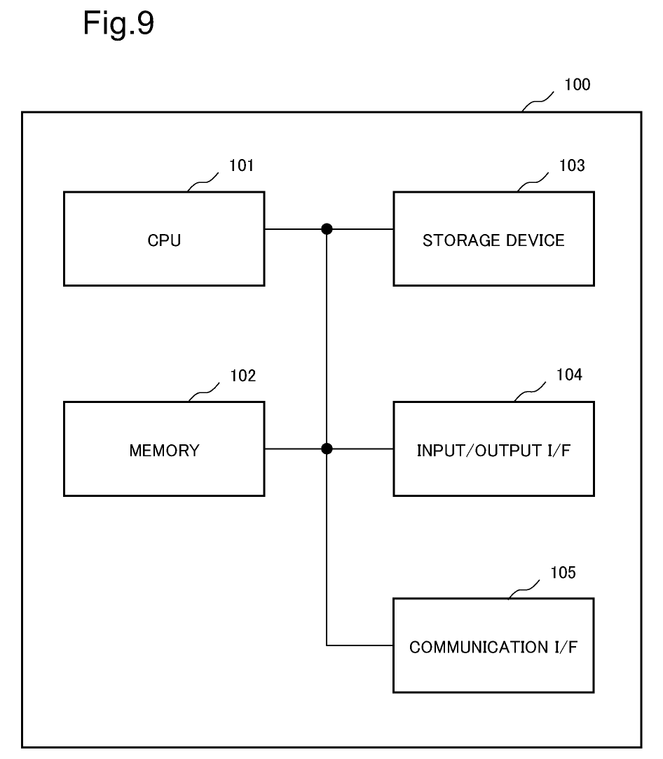

Each processing in the data generation device 10 can be implemented by executing a computer program on a computer. FIG. 9 illustrates an example of a configuration of a computer 100 that executes a computer program for executing each processing in the data generation device 10. The computer 100 includes a central processing unit (CPU) 101, a memory 102, a storage device 103, an input/output interface (I/F) 104, and a communication I/F 105.

The CPU 101 reads and executes the computer program for executing each processing from the storage device 103. The CPU 101 may be configured by a combination of a plurality of CPUs. The CPU 101 may be configured by a combination of a CPU and another type of processor. For example, the CPU 101 may be configured by a combination of a CPU and a graphics processing unit (GPU). The memory 102 includes a dynamic random access memory (DRAM) or the like, and temporarily stores the computer program executed by the CPU 101 and data being processed. The storage device 103 stores the computer program executed by the CPU 101. The storage device 103 includes, for example, a non-volatile semiconductor storage device. The storage device 103 may include another storage device such as a hard disk drive. The input/output I/F 104 is an interface that receives an input from an operator to output display data and the like. The communication I/F 105 is an interface that transmits and receives data to and from the terminal device 20, the data management device 30, and other information processing apparatuses. The terminal device 20 and the data management device 30 can also be configured as in the computer 100.

The computer program used for executing each processing can also be distributed by being stored in a computer-readable recording medium that non-transiently records data. The recording medium can include, for example, a magnetic tape for data recording or a magnetic disk such as a hard disk. The recording medium may include an optical disk such as a compact disc read only memory (CD-ROM). A non-volatile semiconductor storage device may be used as a recording medium.

Time-series data regarding a patient's medical condition may be used for planning a clinical trial plan in a pharmaceutical company. The time-series data regarding the patient’s medical condition may be used for treatment in a medical institution. Such time-series data regarding the patient’s condition is also referred to as a patient journey. For example, reference to time-series data regarding the patient’s medical condition for each case may allow for more accurate planning. Such data used for making a plan regarding the patient may be generated using an information processing system.

However, in the technique described in the prior art, it may be difficult to generate time-series data regarding a medical condition of a patient.

In order to solve the above problems, an object of the present disclosure is to provide a data generation device and the like capable of easily generating time-series data regarding the medical condition of a patient.

According to the present disclosure, it is possible to easily generate time-series data regarding a medical condition of a patient.

Some or all of the above-described example embodiments may be described as the following Supplementary Notes, but are not limited to the following.

### [Supplementary Note 1]

A data generation device including: an acquisition unit that acquires data regarding treatment of a patient; a virtual patient data generation unit that generates data regarding treatment in each stage of a medical condition of a virtual patient based on the data regarding treatment of the patient; a time-series data generation unit that generates time-series data regarding a medical condition of the virtual patient from the generated data regarding treatment of the virtual patient; and an output unit that outputs the generated time-series data regarding the medical condition of the virtual patient.

### [Supplementary Note 2]

The data generation device according to Supplementary Note 1, in which the virtual patient data generation unit generates data regarding treatment of each of the virtual patients using a generative model that generates data regarding treatment of the virtual patient from data regarding treatment.

### [Supplementary Note 3]

The data generation device according to Supplementary Note 2, in which the virtual patient data generation unit generates data of an electronic medical record of the virtual patient as data regarding treatment of the virtual patient using the generative model based on data of an electronic medical record of the patient which is data regarding treatment of the patient.

### [Supplementary Note 4]

The data generation device according to any one of Supplementary Notes 1 to 3, in which the virtual patient data generation unit generates data regarding complications of the virtual patient as data regarding treatment of the virtual patient.

### [Supplementary Note 5]

The data generation device according to any one of Supplementary Notes 1 to 4, further including a selection unit that selects data suitable as target disease data among the generated data regarding treatment of the virtual patient.

### [Supplementary Note 6]

The data generation device according to any one of Supplementary Notes 1 to 5, in which the acquisition unit further acquires data regarding a health condition of a patient before visiting a hospital as data regarding treatment of the patient, and the virtual patient data generation unit generates data regarding treatment of the virtual patient in each stage of the medical condition including a patient’s condition before visiting a hospital.

### [Supplementary Note 7]

The data generation device according to any one of Supplementary Notes 1 to 6, in which the acquisition unit further acquires data regarding a health condition of the patient after discharge as data regarding treatment of the patient, and the virtual patient data generation unit generates data regarding treatment in each stage of the medical condition of the virtual patient, the medical condition including a patient’s condition after discharge.

### [Supplementary Note 8]

The data generation device according to any one of Supplementary Notes 1 to 7, further including a prediction unit that predicts, by using a machine learning model that predicts, based on data regarding treatment in an initial stage of a subject patient, data regarding treatment in a stage after the initial stage on a time series from data in the initial stage of the patient, data regarding treatment of the subject patient in a stage after the initial stage.

### [Supplementary Note 9]

The data generation device according to any one of Supplementary Notes 1 to 8, in which the data regarding the treatment includes recognition of a medical condition by a patient.

### [Supplementary Note 10]

The data generation device according to any one of Supplementary Notes 1 to 9, in which time-series data regarding a medical condition of the virtual patient is a patient journey.

### [Supplementary Note 11]

The data generation device according to Supplementary Note 2 or 3, in which the generative model is a large-scale language model.

### [Supplementary Note 12]

The data generation device according to Supplementary Note 6, in which the acquisition unit acquires data regarding treatment of a patient in a case where the patient visits another hospital as data regarding a health condition of the patient before visiting a hospital.

### [Supplementary Note 13]

A data generation method including: acquiring data regarding treatment of a patient; generating data regarding treatment in each stage of a medical condition of a virtual patient based on the data regarding treatment of the patient; generating time-series data regarding a medical condition of the virtual patient from the generated data regarding treatment of the virtual patient; and outputting the generated time-series data regarding the medical condition of the virtual patient.

### [Supplementary Note 14]

A non-transitory recording medium recording a program for causing a computer to execute: a process of acquiring data regarding treatment of a patient; a process of generating data regarding treatment in each stage of a medical condition of a virtual patient based on the data regarding treatment of the patient; a process of generating time-series data regarding a medical condition of the virtual patient from the generated data regarding treatment of the virtual patient; and a process of outputting the generated time-series data regarding the medical condition of the virtual patient.

Some or all of the configurations described in Supplementary Notes 2 to 12 dependent on the above-described Supplementary Note 1 can also be dependent on Supplementary Notes 13 and 14 by the same dependency relationship as in Supplementary Notes 2 to 12. Some or all of the configurations described as the Supplementary Notes can be similarly dependent on not only the Supplementary Notes 1, 13, and 14, but also various pieces of hardware and software, and various recording means or systems for recording software without departing from the above-described example embodiments.

The previous description of embodiments is provided to enable a person skilled in the art to make and use the present disclosure. Moreover, various modifications to these example embodiments will be readily apparent to those skilled in the art, and the generic principles and specific examples defined herein may be applied to other embodiments without the use of inventive faculty. Therefore, the present disclosure is not intended to be limited to the example embodiments described herein but is to be accorded the widest scope as defined by the limitations of the claims and equivalents.

Further, it is noted that the inventor's intent is to retain all equivalents of the claimed invention even if the claims are amended during prosecution.

1. A data generation device comprising:
at least one memory storing instructions; and
at least one processor configured to access the at least one memory and execute the instructions to:
acquire an electronic medical record that includes data regarding treatment of a patient;
generate data of an electronic medical record of a virtual patient as data regarding treatment of the patient, based on the electronic medical record that includes the data regarding treatment of the patient, wherein
the electronic medical record of the virtual patient includes data regarding treatment in each stage of a medical condition of the virtual patient, and
the data of an electronic medical record of a virtual patient is generated by using a generative model that generates data regarding treatment of the virtual patient from data regarding treatment of the patient;
generate time-series data regarding a medical condition of the virtual patient from the generated data regarding treatment of the virtual patient; and
output the generated time-series data regarding the medical condition of the virtual patient.

2. The data generation device according to claim 1, wherein
the at least one processor is further configured to execute the instructions to:
generate data regarding complications of the virtual patient as data regarding treatment of the virtual patient.

3. The data generation device according to claim 1, wherein
the at least one processor is further configured to execute the instructions to:
select data suitable as target disease data among the generated data regarding treatment of the virtual patient.

4. The data generation device according to claim 1, wherein
the at least one processor is further configured to execute the instructions to:
acquire data regarding a health condition of a patient before visiting a hospital as data regarding treatment of the patient; and
generate data regarding treatment of the virtual patient in each stage of the medical condition including a patient’s condition before visiting a hospital.

5. The data generation device according to claim 1, wherein
the at least one processor is further configured to execute the instructions to:
acquire data regarding a health condition of the patient after discharge as data regarding treatment of the patient; and
generate data regarding treatment in each stage of the medical condition of the virtual patient, the medical condition including a patient’s condition after discharge.

6. The data generation device according to claim 1, wherein
the at least one processor is further configured to execute the instructions to:
predict, by using a machine learning model that predicts, based on data regarding treatment in an initial stage of a subject patient, data regarding treatment in a stage after the initial stage on a time series from data in the initial stage of the patient, data regarding treatment of the subject patient in a stage after the initial stage.

7. The data generation device according to claim 1, wherein
the data regarding the treatment includes recognition of a medical condition by a patient.

8. The data generation device according to claim 1, wherein
time-series data regarding a medical condition of the virtual patient is a patient journey.

9. The data generation device according to claim 1, wherein
the generative model is a large-scale language model.

10. The data generation device according to claim 4, wherein
the at least one processor is further configured to execute the instructions to:
acquire data regarding treatment of a patient in a case where the patient visits another hospital as data regarding a health condition of the patient before visiting a hospital.

11. A data generation method comprising:
acquiring an electronic medical record that includes data regarding treatment of a patient;
generating data of an electronic medical record of a virtual patient as data regarding treatment of the patient, based the electronic medical record that includes the data regarding treatment of the patient, wherein
the electronic medical record of the virtual patient includes data regarding treatment in each stage of a medical condition of the virtual patient, and
the data of an electronic medical record of a virtual patient is generated by using a generative model that generates data regarding treatment of the virtual patient from data regarding treatment of the patient;
generating time-series data regarding a medical condition of the virtual patient from the generated data regarding treatment of the virtual patient; and
outputting the generated time-series data regarding the medical condition of the virtual patient.

12. A non-transitory recording medium recording a program for causing a computer to execute:
a process of acquiring an electronic medical record that includes data regarding treatment of a patient;
a process of generating data of an electronic medical record of a virtual patient as data regarding treatment of the patient, based on the electronic medical record that includes the data regarding treatment of the patient, wherein
the electronic medical record of the virtual patient includes data regarding treatment in each stage of a medical condition of the virtual patient, and
the data of an electronic medical record of a virtual patient is generated by using a generative model that generates data regarding treatment of the virtual patient from data regarding treatment of the patient;
a process of generating time-series data regarding a medical condition of the virtual patient from the generated data regarding treatment of the virtual patient; and
a process of outputting the generated time-series data regarding the medical condition of the virtual patient.
